(19) United States
(12) Patent Application Publication
(10) Pub. No.: US 2026/0069179 A1
(43) Pub. Date: Mar. 12, 2026

(54) SYSTEM AND METHOD FOR SCALABLE MULTIMODAL THEORY-OF-MIND REASONING

(71) Applicant: Honda Motor Co., Ltd., Tokyo (JP)

(72) Inventors: Chunhui ZHANG, Lebanon, NH (US); Shao-Yuan LO, Milpitas, CA (US); Kwonjoon LEE, Sunnyvale, CA (US); Nakul AGARWAL, San Francisco, CA (US)

(21) Appl. No.: 19/084,518

(22) Filed: Mar. 19, 2025

Related U.S. Application Data
(60) Provisional application No. 63/692,586, filed on Sep. 9, 2024.

(51) Int. Cl. A61B 5/16; G05B 19/02; G06V 40/20

(52) U.S. Cl. CPC A61B 5/165; G05B 19/02; G06V 40/20

(57) ABSTRACT

A system for inferring human mental states through a multimodal theory-of-mind (ToM) framework includes a processor and a memory. The memory stores instructions that when executed by the processor cause the processor to perform the following. The processor receives a video dataset and a textual dataset associated with an environmental scene. The processor converts at least a portion of the video dataset and the textual dataset into symbolic representations. Based on the symbolic representations, the processor generates action-likelihood distributions via a language model based policy obtained by combining a large pre-trained language model with a smaller post-trained language model through a weak-to-strong control mechanism. The processor applies a Bayesian inverse-planning procedure that generates posterior probabilities over multiple goal-belief hypotheses based on the action-likelihood distributions and the symbolic representations. The processor outputs at least one inferred mental state of the agent by selecting from among the multiple goal-belief hypotheses.

### BACKGROUND

Human-like social cognition involves “Theory of Mind” (ToM)—the capacity to infer and predict others' thoughts, emotions, intentions, and beliefs. Machine reasoning based on ToM can improve numerous applications, including human-computer interaction, embodied robotics, and other systems requiring nuanced understanding of human mental states. However, many existing solutions either rely on language models (LMs) that demand extensive domain-specific fine tuning, or they employ purely Bayesian approaches that lack scalability across diverse scenarios.

Recent work in multimodal ToM reasoning integrates Bayesian methods for belief-goal inference with neural network modules handling both visual and textual inputs. Although post-training smaller LMs for TOM tasks is generally feasible, extending the same approach to much larger LMs (e.g., 70B or 405B parameters) becomes prohibitively expensive. Moreover, smaller LMs may have limited world knowledge or insufficient reasoning depth. Accordingly, there is a need for a weak-to-strong control mechanism in which a post-trained small LM can guide a large LM, thereby preserving scalability while maintaining contextual alignment for ToM tasks.

### BRIEF DESCRIPTION

According to one aspect, a computer-implemented method for inferring human mental states through a multimodal theory-of-mind (TOM) framework is provided. The method includes receiving a video dataset and a textual dataset associated with an environmental scene. The method includes converting at least a portion of the video dataset and the textual dataset into symbolic representations of states, actions, and hypotheses related to an agent's goals and beliefs. The method includes generating, based on the symbolic representations, action-likelihood distributions via a language model based policy. The language model based policy is obtained by combining a large pre-trained language model with a smaller post-trained language model through a weak-to-strong control mechanism. The method includes applying a Bayesian inverse-planning procedure that generates posterior probabilities over multiple goal-belief hypotheses based on the action-likelihood distributions and the symbolic representations. The method includes outputting at least one inferred mental state of the agent by selecting from among the multiple goal-belief hypotheses.

According to another aspect, a system for inferring human mental states through a multimodal theory-of-mind (ToM) framework includes a processor and a memory. The memory stores instructions that when executed by the processor cause the processor to perform the following. The processor receives a video dataset and a textual dataset associated with an environmental scene. The processor converts at least a portion of the video dataset and the textual dataset into symbolic representations of states, actions, and hypotheses related to an agent's goals and beliefs. The processor generates, based on the symbolic representations, action-likelihood distributions via a language model based policy. The language model based policy is obtained by combining a large pre-trained language model with a smaller post-trained language model through a weak-to-strong control mechanism. The processor applies a Bayesian inverse-planning procedure that generates posterior probabilities over multiple goal-belief hypotheses based on the action-likelihood distributions and the symbolic representations. The processor outputs at least one inferred mental state of the agent by selecting from among the multiple goal-belief hypotheses.

According to yet another aspect, a non-transitory computer readable storage medium stores instructions that when executed by a computer, which includes a processor performs a method for inferring human mental states through a multimodal theory-of-mind (ToM) framework. The method includes receiving a video dataset and a textual dataset associated with an environmental scene. The method includes converting at least a portion of the video dataset and the textual dataset into symbolic representations of states, actions, and hypotheses related to an agent's goals and beliefs. The method includes generating, based on the symbolic representations, action-likelihood distributions via a language model based policy. The language model based policy is obtained by combining a large pre-trained language model with a smaller post-trained language model through a weak-to-strong control mechanism. The method includes applying a Bayesian inverse-planning procedure that generates posterior probabilities over multiple goal-belief hypotheses based on the action-likelihood distributions and the symbolic representations. The method includes outputting at least one inferred mental state of the agent by selecting from among the multiple goal-belief hypotheses.

### BRIEF DESCRIPTION OF THE DRAWINGS

The novel features believed to be characteristic of the disclosure are set forth in the appended claims. In the descriptions that follow, like parts are marked throughout the specification and drawings with the same reference numerals, respectively. The drawing figures are not necessarily drawn to scale and certain figures can be shown in exaggerated or generalized form in the interest of clarity and conciseness. The disclosure itself, however, as well as a preferred mode of use, further objects and advances thereof, will be best understood by reference to the following detailed description of exemplary embodiments when read in conjunction with the accompanying drawings.

FIG. 1 is a schematic view of an exemplary operating environment for inferring human mental states through a multimodal ToM framework according to an exemplary embodiment of the present disclosure.

FIG. 2 is a schematic overview of a multimodal ToM reasoning application according to an exemplary embodiment of the present disclosure.

FIG. 3 is an illustrative framework that is executed by the multimodal TOM reasoning application for inferring human mental states according to an exemplary embodiment of the present disclosure.

FIG. 4 is a process flow diagram of a method of inferring and applying human mental states through a multimodal ToM framework according to an exemplary embodiment of the present disclosure.

FIG. 5 is a process flow diagram of a method for inferring human mental states through multimodal ToM according to an exemplary embodiment of the present disclosure.

### DETAILED DESCRIPTION

The following includes definitions of selected terms employed herein. The definitions include various examples and/or forms of components that fall within the scope of a term and that may be used for implementation. The examples are not intended to be limiting. Further, one having ordinary skill in the art will appreciate that the components discussed herein, may be combined, omitted, or organized with other components or organized into different architectures.

A “bus”, as used herein, refers to an interconnected architecture that is operably connected to other computer components inside a computer or between computers. The bus may transfer data between the computer components. The bus may be a memory bus, a memory controller, a peripheral bus, an external bus, a crossbar switch, and/or a local bus, among others. The bus can also be a vehicle bus that interconnects components inside a vehicle using protocols such as Media Oriented Systems Transport (MOST), Controller Area network (CAN), Local Interconnect Network (LIN), among others.

A “computer communication”, as used herein, refers to a communication between two or more computing devices (e.g., computer, personal digital assistant, cellular telephone, network device) and can be, for example, a network transfer, a file transfer, an applet transfer, an email, a hypertext transfer protocol (HTTP) transfer, and so on. A computer communication can occur across, for example, a wireless system (e.g., IEEE 802.11), an Ethernet system (e.g., IEEE 802.3), a token ring system (e.g., IEEE 802.5), a local area network (LAN), a wide area network (WAN), a point-to-point system, a circuit switching system, a packet switching system, among others.

A “disk”, as used herein can be, for example, a magnetic disk drive, a solid-state disk drive, a floppy disk drive, a tape drive, a Zip drive, a flash memory card, and/or a memory stick. Furthermore, the disk can be a CD-ROM (compact disk ROM), a CD recordable drive (CD-R drive), a CD rewritable drive (CD-RW drive), and/or a digital video ROM drive (DVD ROM). The disk can store an operating system that controls or allocates resources of a computing device.

A “memory”, as used herein can include volatile memory and/or non-volatile memory. Non-volatile memory can include, for example, ROM (read only memory), PROM (programmable read only memory), EPROM (erasable PROM), and EEPROM (electrically erasable PROM). Volatile memory can include, for example, RAM (random access memory), synchronous RAM (SRAM), dynamic RAM (DRAM), synchronous DRAM (SDRAM), double data rate SDRAM (DDR SDRAM), and direct RAM bus RAM (DRRAM). The memory can store an operating system that controls or allocates resources of a computing device.

A “module”, as used herein, includes, but is not limited to, non-transitory computer readable medium that stores instructions, instructions in execution on a machine, hardware, firmware, software in execution on a machine, and/or combinations of each to perform a function(s) or an action(s), and/or to cause a function or action from another module, method, and/or system. A module may also include logic, a software-controlled microprocessor, a discreet logic circuit, an analog circuit, a digital circuit, a programmed logic device, a memory device containing executing instructions, logic gates, a combination of gates, and/or other circuit components. Multiple modules may be combined into one module and single modules may be distributed among multiple modules.

An “operable connection”, or a connection by which entities are “operably connected”, is one in which signals, physical communications, and/or logical communications may be sent and/or received. An operable connection may include a wireless interface, a physical interface, a data interface and/or an electrical interface.

A “processor”, as used herein, processes signals and performs general computing and arithmetic functions. Signals processed by the processor may include digital signals, data signals, computer instructions, processor instructions, messages, a bit, a bit stream, or other means that may be received, transmitted and/or detected. Generally, the processor may be a variety of various processors including multiple single and multicore processors and co-processors and other multiple single and multicore processor and co-processor architectures. The processor may include various modules to execute various functions.

A “value” and “level”, as used herein may include, but is not limited to, a numerical or other kind of value or level such as a percentage, a non-numerical value, a discrete state, a discrete value, a continuous value, among others. The term “value of X” or “level of X” as used throughout this detailed description and in the claims refers to any numerical or other kind of value for distinguishing between two or more states of X. For example, in some cases, the value or level of X may be given as a percentage between 0% and 100%. In other cases, the value or level of X could be a value in the range between 1 and 10. In still other cases, the value or level of X may not be a numerical value, but could be associated with a given discrete state, such as “not X”, “slightly x”, “x”, “very x” and “extremely x”.

A “database”, as used herein, may refer to a table, a set of tables, and a set of data stores (e.g., disks) and/or methods for accessing and/or manipulating those data stores.

A “mobile device”, as used herein, may be a computing device typically having a display screen with a user input (e.g., touch, keyboard) and a processor for computing. Mobile devices include handheld devices, portable electronic devices, smart phones, laptops, tablets, and e-readers.

A “multi-stream input”, as used herein, may be multiple data sources or streams that feed into a model.

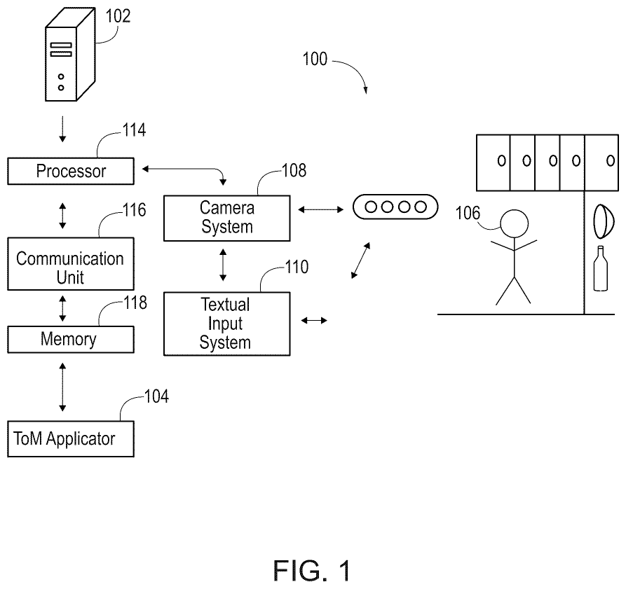

Referring now to the drawings, where the showings are for purposes of illustrating one or more exemplary embodiments and not for purposes of limiting the same, FIG. 1 is a schematic view of an exemplary operating environment 100 for inferring human mental states through a multimodal TOM framework according to an exemplary embodiment of the present disclosure. The operating environment 100 includes a system that provides for the computer-implemented execution of a framework to infer human mental states (e.g., goals and beliefs) based on both video and textual information.

In an exemplary embodiment, the operating environment 100 may include an externally hosted server infrastructure (external server) 102 that is configured to execute a multimodal theory-of-mind application (TOM application) 104. As discussed in more detail below, the TOM application 104 may be configured to execute processes that allow the generation of symbolic representations from raw video streams and textual descriptions, followed by performing a Bayesian inverse-planning procedure using one or more language models to infer the mental states of an observed agent 106.

As shown in FIG. 1, the TOM application 104 may be configured to receive video data and textual data associated with the agent 106 performing one or more actions within an environment. In the exemplary embodiment of FIG. 1, the agent 106 is shown as a person, but it is to be appreciated that the agent 106 may include any type of entity (e.g., a humanoid robot, a human user, a virtual avatar, etc.) whose goals or beliefs are to be inferred. The ToM application 104 may convert the video data, for example by extracting scene information and object relationships from each frame of the video data, into a set of symbolic predicates. It may also parse the textual data to produce symbolic representations of the environment's states, the agent's 106 potential goals or beliefs, as well as any relevant timestamps or actions. By merging these video and text derived symbols into a unified representation, the ToM application 104 obtains a structured view of the initial states of the environment, subsequent or observed actions within the environment, and hypotheses concerning the agent's 106 mental states.

As discussed in more detail below, the TOM application 104 may receive the video data (e.g., image frames) via a camera system 108 that can be operably connected to, or wirelessly communicate with, the external server 102. In alternative exemplary embodiments, the TOM application 104 may obtain video data through existing or stored video files, rather than live camera feeds. The TOM application 104 may also receive textual data (e.g., scripts of agent actions, environment descriptions, or instructions) provided by a textual input system 110 that is similarly operably connected to or in communication with the external server 102. In one embodiment, the textual input system 110 can include an automated parser or natural language interface that streams textual context describing the agent 106 and its environment, potentially in operable connection with the video feed from the camera system 108.

Upon receiving the video data and the textual data, the TOM application 104 may be configured to execute a process that combines symbolic representations of the environment states and actions with a Bayesian inverse-planning procedure. In one or more embodiments, the TOM application 104 may also be configured to incorporate probability updates for the agent's 106 belief state, enabling incremental inference of goals and beliefs across multiple timesteps.

As discussed below, for each set of observed actions, the TOM application 104 may be configured to estimate action-likelihood distributions via a language model (LM) based policy. In particular, the TOM application 104 may employ a large pre-trained LM that is reweighted by a smaller, post-trained LM through a weak-to-strong control mechanism. The smaller LM is specialized for the given domain or scenario, while the large LM brings extensive world knowledge. By reweighting the large LM's policy distribution according to the ratio of the post-trained LM over a naive small LM, the ToM application 104 can achieve effective, scalable Bayesian inference of mental states.

In an exemplary embodiment, the TOM application 104 may generate multiple posterior hypotheses regarding the agent's 106 goals or beliefs by applying a structured Bayesian update. More specifically, the TOM application 104 compares each hypothesis using a cumulative log-likelihood derived from the agent's 106 observed actions and symbolic environment states. The hypothesis with the highest cumulative log-likelihood is deemed more likely to reflect the agent's 106 actual goal or belief. Once the TOM application 104 infers the agent's 106 mental state, it may pass this information to downstream systems, for example, to refine human-computer interactions, simulate human-robot collaboration, improve conversation policies, or perform autonomous control of an autonomous system within an interactive environment.

In one or more embodiments, the TOM application 104 may be further configured to send commands to externally hosted electronic components (not shown) once it concludes that the agent 106 holds a particular goal or belief. For instance, if the inferred mental state reveals the agent's 106 intention to retrieve an object, the ToM application 104 can trigger an autonomous robot control system (not shown) to assist. In another exemplary embodiment, the TOM application 104 may transmit real-time prompts or clarifications to a user interface system based on the agent's 106 inferred mental state, thereby enhancing collaborative decision-making or user guidance.

Additionally, in an exemplary embodiment, the TOM application 104 can provide adaptive assistance to user-facing software-such as prompting a user with contextually relevant information when it infers the user is operating under a false belief. It is to be appreciated that these scenarios are merely non-limiting examples of how the TOM application 104 can generate commands or messages based on inferred mental states. Many alternative embodiments exist, encompassing social computing platforms, assistive technologies, or interactive simulations-all of which fall within the scope of this disclosure.

The TOM application 104 can improve the field of multimodal social intelligence by enabling interpretation of an agent's 106 behavior in both video and text and infers mental states without relying on exhaustive or purely end-to-end training on large, specialized datasets. Its functionality can integrate symbolic state representations with large-scale language models under a Bayesian inverse planning framework, leading to more robust, cost-effective, and interpretable inference of goals and beliefs, even in dynamic or unstructured environments.

With continued reference to FIG. 1, the external server 102 may be operably controlled by a processor 114 configured to execute the TOM application 104. The processor 114 may run the necessary operating systems, database engines, and other software modules, and may include internal memory, an interface circuit, and bus lines for managing data transfers, sending commands, and communicating with multiple components of the external server 102.

The processor 114 may be operably connected to a communication unit 116, which may include one or more network interfaces (not shown) configured to connect to other computing systems via a network cloud (not shown). The communication unit 116 can support secure communications between the external server 102 and, for example, the camera system 108, the textual input system 110, or any other devices that may receive output (e.g., commands or inferred mental states) from the TOM application 104.

In one embodiment, the processor 114 may be operably connected to a memory 118 within the external server 102. The processor 114 can retrieve and execute various software components from the memory 118, such as the TOM application 104 and related LM modules. By way of example, the memory 118 may hold both the large pre-trained LM and one or more smaller LM's, as well as program instructions enabling the Bayesian inference procedures.

In an exemplary embodiment, the smaller, post-trained LM is used for domain-specific adaptation (e.g., environment or user-oriented tasks), while the large pre-trained LM maintains extensive general knowledge. In some embodiments, the smaller, post-trained LM may range from a few hundred million to several billion parameters, while the larger, pre-trained LM may include tens or hundreds of billions of parameters. These ranges are non-limiting examples that reflect typical resource constraints and performance trade-offs in contemporary implementations. To combine their strengths, the TOM application 104 can instantiate a ratio-based reweighting procedure, blending the large LM's policy distribution with that of the post-trained small LM while compensating for a baseline naive small LM. This orchestrated approach can refine the action-likelihood distributions at each observation step, thereby improving interpretability and scalability in inferring the agent's 106 mental state.

Components of the TOM application 104 will now be described according to an exemplary embodiment and with continued reference to FIG. 1. In some implementations, the TOM application 104 may reside in the memory 118 of the external server 102 and is executed by the processor 114. Alternatively, in another embodiment, the TOM application 104 may be hosted externally on a different computing system (not shown) located outside of the external server 102, with the communication unit 116 providing access for the processor 114 to run the ToM application 104 remotely.

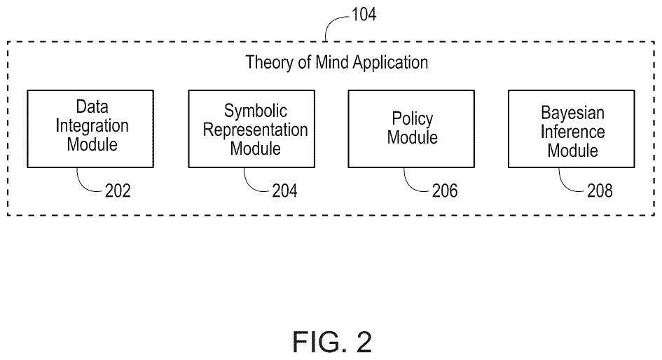

FIG. 2 is a schematic overview of the TOM application 104 according to an exemplary embodiment of the present disclosure. In some implementations, the TOM application 104 may include one or more modules 202-208 that perform multimodal ToM reasoning by way of the processor 114. By way of example, these modules may include: a data integration module 202, responsible for merging or aligning the video data and the textual data; a symbolic representation module 204, which transforms the integrated data into structured predicates; a policy module 206, configured to generate action-likelihood distributions (e.g., via large and small language models); and a Bayesian inference module 208, which updates and compares hypotheses about the agent's 106 goals or beliefs. It is appreciated that the TOM application 104 may include additional modules and/or submodules in lieu of or in addition to the modules 202-208.

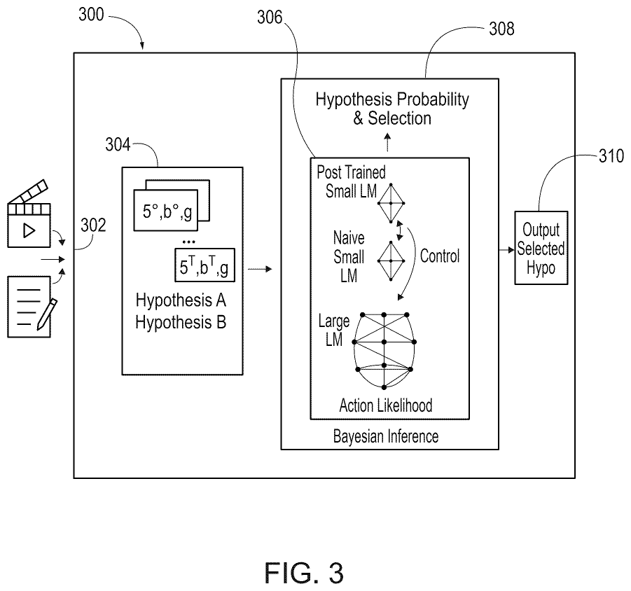

FIG. 3 depicts an illustrative framework 300 executed by the TOM application 104 for inferring human mental states from multimodal data, in accordance with an exemplary embodiment of the present disclosure. In this example, the camera system 108 captures video of the agent 106, while the textual input system 110 provides environment descriptions, agent action scripts, or other contextual text. At 302, the camera system 108 and textual input system 110 each communicate their data to the data integration module 202 of the TOM application 104, which synchronizes these inputs by timestamp or event. The integrated data may then be forwarded to the symbolic representation module 204 for further processing.

Upon receiving the video data and the textual information, the data integration module 202 consolidates these multimodal inputs by aligning them based on timestamps or other detected events. For instance, each frame of the video data may be paired with a corresponding time-stamped textual description. The data integration module 202 may also invoke perception routines to convert raw video into a voxel map or identify object bounding boxes, producing a scene graph that captures spatial relationships and the agent's 106 actions. Simultaneously, textual data may be parsed into discrete segments such as environment states, agent instructions, or user queries. The data integration module 202 may then merge these streams into a unified data structure at 304 that captures what was observed, where, and when-thereby setting the stage for further processing.

As shown at 302 and 304, the symbolic representation module may 204 receive this merged, time-aligned data from the data integration module 202 and convert it into symbolic predicates that describe states, actions, object relationships, and relevant timestamps. For example, if the scene graph indicates that the agent 106 opens a fridge at time t, while the textual data states “the pear is inside the basket,” both pieces of information are converted into predicates such as open (agent, fridge, t) and Belief (agent, location=“basket”, object=“pear”). By transforming raw observations into a coherent, queryable format, the symbolic representation module 204 creates a structured view of the environment and the hypothesized mental states.

At 304, the symbolic representation produced by the symbolic representation module 204 may be passed to the policy module 206, which, at 306, is responsible for generating action-likelihood distributions using a LM based policy. In an exemplary embodiment, the policy module 206 employs a “weak-to-strong” control mechanism that combines a large pre-trained LM, offering broad world knowledge, and a smaller post-trained LM, specialized for the current ToM domain. The policy module 206 reweights the large LM's baseline distribution according to the ratio of the specialized small LM's likelihoods over those of a naive small LM—thus transferring post-trained ToM behaviors to the larger LM at inference time. This yields action-likelihood distributions conditioned on candidate goals, beliefs, and states, effectively reflecting how likely a given action sequence is if the agent 106 holds a particular mental state.

At 308, the Bayesian inference module 208 may apply a Bayesian inverse-planning procedure to maintain and update posterior probabilities over multiple goal-belief hypotheses. Specifically, the inference module compares how well each hypothesis explains the observed action sequence—using the action-likelihood distributions from the policy module 206—and incorporates belief-updating details from the symbolic representation (e.g., frames showing the agent 106 picking up an object). By incrementally multiplying or summing log-likelihoods across time, the Bayesian inference module 208 converges on a posterior distribution that identifies the most plausible mental states for the agent 106.

Additionally, at 308, the Bayesian inference module 208 may select which goal-belief hypothesis (or set of hypotheses) aligns best with the combined evidence from the observed actions and symbolic representation. The TOM application 104, at 310, may then output at least one inferred mental state of the agent 106—for instance, “the agent believes the pear is in the basket but is actually in the fridge,” or “the agent intends to retrieve the apple from the cabinet.” This output can be passed to downstream systems (e.g., interactive applications, collaborative robots, or higher-level decision-making processes). Because the TOM application 104 integrates scene-graph-based visual inputs, textual context, and a scalable LM-based policy, it can accurately infer human mental states in complex, multimodal environments without having to post-train large models directly.

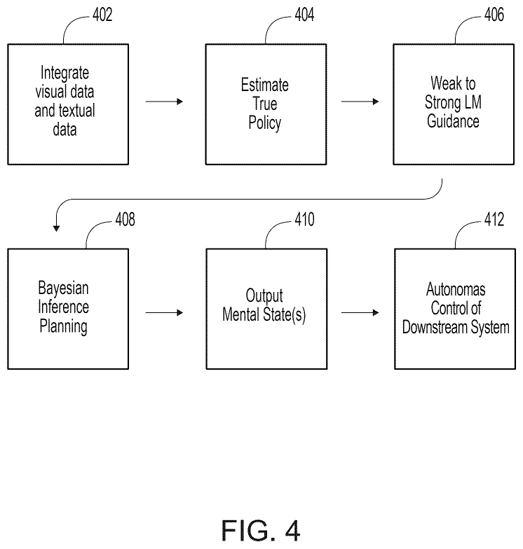

FIG. 4 is a process flow diagram of a method of inferring and applying human mental states through a multimodal ToM framework according to an exemplary embodiment of the present disclosure. FIG. 4 is described with partial reference to FIGS. 1-3, and it should be appreciated that method 400 may be carried out using the modules 202-208 described above, alternate architectures, or other suitable components.

At 402, the method 400 includes integrating received visual and textual data into a unified symbolic representation of the agent's 106 context and behavior. To achieve this, visual data is fused—converted into symbolic predicates (e.g., by generating a voxel map and scene graph for each video frame)—with textual data, which is parsed to produce symbolic representations of the initial state, subsequent actions, and any relevant questions. For instance, a textual parser—such as a natural language processing (NLP) pipeline, a rule-based parser, and/or a deep learning-based model—may extract structured information from descriptions, such as environmental details (“the pear is inside the basket”), actions (“walks toward the kitchen”), or potential goals and beliefs (“retrieve the pear” vs. “the pear is not inside the basket”). These multimodal streams may then be aligned over time, creating a symbolic state that captures the agent's 106 environment and possible objectives.

At 404, the method 400 includes estimating a true policy in order to generate action likelihoods. The true policy π(a^{τ} | g, b^{τ}) is estimated through a language model based probability \tilde{π} (a^{t} | s^{t}, g, \hat{b}^{t}) plus an error term ε denoting an approximation error during estimation:

$$π ( {a}^{τ} \vert g , {b}^{τ} ) = \tilde{π} ( {a}^{t} \vert {s}^{t} , g , {\hat{b}}^{t} ) + ε \qquad ( 1 )$$

When substituted into a posterior probability framework (i.e., Bayesian inverse planning to be described), it yields:

$$( 2 )$$

$$P ( g , {b}^{t} \vert {s}^{1 : t} , {a}^{1 : t - 1} ) ∝ {\prod }_{τ = 1}^{t} [ \tilde{π} ( {a}^{τ} \vert {s}^{τ} , g , {\hat{b}}^{τ} ) + ε ] \cdot P ( {b}^{τ} \vert {b}^{τ - 1} , {s}^{τ} ) \cdot P ( {b}^{0} ) P ( g )$$

Equation (2) shows how the LM estimated policy \tilde{π} can be plugged into a Bayesian framework, accounting for actions, states, and belief updates over time. In application, reducing ε may require post-training the LM to align its pretrained knowledge to the current scenario. In this regard, and still at 404, the post-training stage (ToM optimization) may occur. The method 400 may refine ε by learning a scenario-specific policy π^{ε} from an action-policy experience pool. This pool D may be defined as:

$$𝒟 = {\{ ( {s}_{i} , {b}_{i} , {g}_{i} , {a}_{i} ) \}}_{i = 1}^{N} ,$$

where sᵢ, bᵢ, gᵢ, and aᵢ denote sequences of states, beliefs, goals, and actions sourced from multimodal situations. The objective function guiding post-training is:

$$ℒ ( {π}^{ε} ) = - {\sum }_{i = 1}^{N} log {π}^{ε} ( {a}_{i} \vert {s}_{i} , {b}_{i} , {g}_{i} ) \qquad ( 3 )$$

Here, π^{ε} learns human TOM behaviors, enabling the LM-based policy to handle complex, multimodal situations. Because post-training is done on smaller models, it remains resource-efficient while achieving alignment with the target domain.

At 406, the method 400 combines the post-trained small LM with a larger LM via weak-to-strong guidance to redirect the larger LM's output distribution. Concretely, at each inference step t, the final policy distribution \hat{π} is:

$$\overline{π} ( {a}^{t} \vert {s}^{t} , g , {\hat{b}}^{t} ) = \frac{1}{\overline{Z}} {π}^{ℒ} ( {a}^{t} \vert {s}^{t} , g , {\hat{b}}^{t} ) \frac{{π}^{ε} ( {a}^{t} \vert {s}^{t} , g , {\hat{b}}^{t} )}{{π}^{𝒩} ( {a}^{t} \vert {s}^{t} , g , {\hat{b}}^{t} )} \qquad ( 4 )$$

where π^{L} denotes the large LM's policy, π^{ε} is the post-trained small LM, and π^{N} is a naive small LM (before post-training). The ratio

$$\frac{{π}^{ε} ( {a}^{t} \vert {s}^{t} , g , {\hat{b}}^{t} )}{{π}^{N} ( {a}^{t} \vert {s}^{t} , g , {\hat{b}}^{t} )}$$

captures how post-training shifts the small LM's behavior, which then redirects the large LM predictions at inference time. The normalization factor Z̅ ensures the distribution remains valid. Overall, this technique transfers ToM behaviors from the small LM to the large LM at test time, scaling Bayesian inference capabilities without incurring the cost of fine-tuning the large model itself.

At 408, a Bayesian inverse planning approach may be applied to generate and evaluate hypotheses about the agent's 106 intentions. These action likelihoods (from the weak-to-strong LM policy) inform posterior probabilities over different mental-state explanations (goals/beliefs).

Still at 408, the method 400 may model the agent's 106 behavior as a forward generative model under a Partially Observable Markov Decision Process (POMDP), defined by the tuple (S, A, [symbol] , G, R, Ω, O, γ). Here, s^{t}∈S and a^{t}∈A represent the state and action at time t, respectively. [symbol] (s^{t} | s, a) denotes the state transition probabilities. The goal g∈G determines the reward r^{t}=R(s^{t}, a^{t}, g). The agent's 106 observation o^{t}∈Ω is obtained via the observation function o^{t}=O(s^{t}). The discount factor is γ∈(0,1]. The agent's 106 belief, b(s), is a probability distribution capturing uncertainty about object locations.

Still at 408, the method 400 may invert this forward model to jointly infer the agent's 106 goals and beliefs from observed states and actions. Under deterministic transitions, the posterior probability of a goal g and belief b^{t}, given the observed state-action sequence, is:

$$P ( g , {b}^{t} \vert {s}^{1 : t} , {a}^{1 : t - 1} ) ∝ {\prod }_{τ = 1}^{t} π ( {a}^{τ} \vert g , {b}^{τ} ) P ( {b}^{τ} \vert {b}^{τ - 1} , {s}^{τ} ) \cdot P ( {b}^{0} ) P ( g ) \qquad ( 5 )$$

Here, π(a^{τ} | g, b^{τ}) represents the policy from the POMDP model, to be replaced by the weak-to-strong distribution π̅ from equation (4). The term P(b^{τ} | b^{τ-1}, s^{τ}) captures how the agent's 106 beliefs evolve based on new observations, directly linking to the POMDP's belief update process. To compare competing hypotheses, consider two competing possibilities

$${H}_{1} = 〈 {g}_{1} , {b}_{1}^{t} 〉 and {H}_{2} = 〈 {g}_{2} , {b}_{2}^{t} 〉 ,$$

representing different goal-belief pairs. Their relative log-likelihoods are compared through the following equation:

$$log \frac{P ( {g}_{1} , {b}_{1}^{t} \vert {s}^{1 : t} , {a}^{1 : t} )}{P ( {g}_{2} , {b}_{2}^{t} \vert {s}^{1 : t} , {a}^{1 : t} )} = {\underbrace{log \frac{π ( {a}^{t} \vert {g}_{1} , {b}_{1}^{t} )}{π ( {a}^{t} \vert {g}_{2} , {b}_{2}^{t} )} + log \frac{P ( {b}_{1}^{t} \vert {\hat{b}}^{t - 1} , {s}^{t} )}{P ( {b}_{2}^{t} \vert {\hat{b}}^{t - 1} , {s}^{t} )}}}_{Current step comparison} + {\underbrace{{\sum }_{τ = 1}^{t - 1} log \frac{π ( {a}^{τ} \vert {g}_{1} , {\hat{b}}^{τ} )}{π ( {a}^{τ} \vert {g}_{2} , {\hat{b}}^{τ} )}}}_{Prior steps comparison} \qquad ( 6 )$$

The first term compares actions and belief updates at the current step, while the second term aggregates prior-step evidence. Thus, the entire action history informs which hypothesis best explains the agent's 106 behavior. This structure ensures that the policy model (including any LM-based approach) is consistently applied across all timesteps for Bayesian inference of the agent's 106 mental states.

At 410, the method 400 may include outputting at least one inferred mental state by selecting from among the hypothesis compared at 408. Based on the at least one inferred mental state, the method may include, at 412, electronically controlling a downstream system or interface—for example, to provide user assistance, coordinate robotic actions, or adjust an interactive learning environment based on the agent's 106 inferred mental state. Such control of the downstream system may be accomplished by the TOM application 104 initiating control commands through a dedicated control module or outputting control instructions.

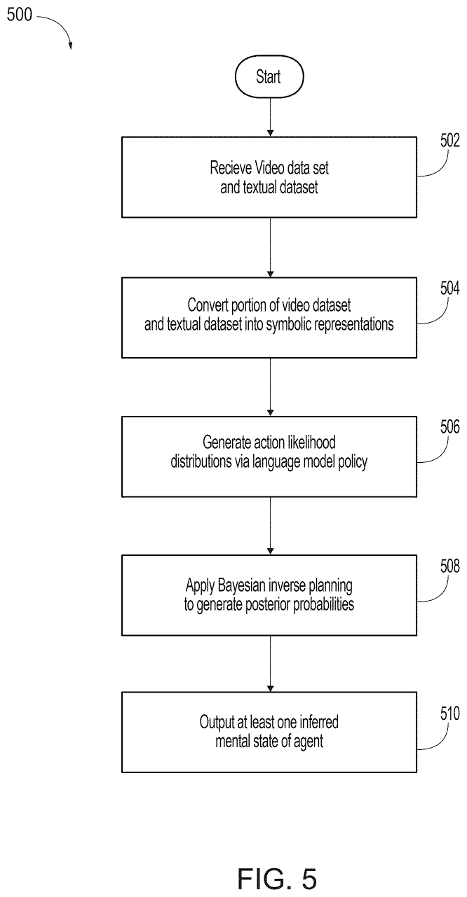

FIG. 5 is a process flow diagram of a method for inferring human mental states through multimodal ToM according to an exemplary embodiment of the present disclosure. FIG. 5 will be described with reference to the components of FIGS. 1-3, though it is to be appreciated that the method 500 of FIG. 5 may be used with other systems/components. At 502, the method 500 may include receiving a video dataset and a textual dataset associated with an environmental scene. At 504, the method 500 may include converting at least a portion of the video dataset and the textual dataset into symbolic representations of states, actions, and hypotheses related to an agent's 106 goals and beliefs. At 506, the method 500 may include generating, based on the symbolic representations, action-likelihood distributions via a language model based policy. The language model based policy may be obtained by combining a large pre-trained language model with a smaller post-trained language model through a weak-to-strong control mechanism. At 508, the method 500 may include applying a Bayesian inverse-planning procedure that generates posterior probabilities over multiple goal-belief hypotheses based on the action-likelihood distributions and the symbolic representations. At 510, the method 500 may include outputting at least one inferred mental state of the agent 106 by selecting from among the multiple goal-belief hypotheses. In one embodiment, the at least one inferred mental state may be utilized to electronically control at least one electronic component to complete at least one task associated with the agent's 106 predicted intent.

As used in this application, the terms “component”, “module”, “system”, “interface”, and the like are generally intended to refer to a computer-related entity, either hardware, a combination of hardware and software, software, or software in execution. For example, a component may be, but is not limited to being, a process running on a processor, a processing unit, an object, an executable, a thread of execution, a program, or a computer. By way of illustration, both an application running on a controller and the controller may be a component. One or more components residing within a process or thread of execution and a component may be localized on one computer or distributed between two or more computers.

Further, the claimed subject matter may be implemented as a method, apparatus, or article of manufacture using standard programming or engineering techniques to produce software, firmware, hardware, or any combination thereof to control a computer to implement the disclosed subject matter. The term “article of manufacture” as used herein is intended to encompass a computer program accessible from any computer-readable device, carrier, or media. Of course, many modifications may be made to this configuration without departing from the scope or spirit of the claimed subject matter.

Generally, aspects are described in the context of “computer readable instructions” being executed by one or more computing devices. Computer readable instructions may be distributed via computer readable media, as discussed below. These instructions can be implemented as program modules, such as functions, objects, Application Programming Interfaces (APIs), data structures, and the like, which perform one or more tasks or implement one or more abstract data types. Typically, the functionality of the computer readable instructions is combined or distributed as desired in various environments and, for example, stored in the memory 118 to be implemented by the processor 114.

The term “computer readable media” as used herein includes computer storage media. Computer storage media includes volatile and nonvolatile, removable, and non-removable media implemented in any method or technology for storage of information such as computer readable instructions or other data. The memory 118 is an example of computer storage media. Computer storage media includes, but is not limited to, RAM, ROM, EEPROM, flash memory or other memory technology, CD-ROM, Digital Versatile Disks (DVDs) or other optical storage, magnetic cassettes, magnetic tape, magnetic disk storage or other magnetic storage devices, or any other medium which may be used to store the desired information and which may be accessed by the processor 114.

The term “computer readable media” includes communication media. Communication media typically embodies computer readable instructions or other data in a “modulated data signal” such as a carrier wave or other transport mechanism and includes any information delivery media. The term “modulated data signal” includes a signal that has one or more of its characteristics set or changed in such a manner as to encode information in the signal.

Although the subject matter has been described in language specific to structural features or methodological acts, it is to be understood that the subject matter of the appended claims is not necessarily limited to the specific features or acts described above. Rather, the specific features and acts described above are disclosed as example aspects.

Various operations of aspects are provided herein. The order in which one or more or all of the operations are described should not be construed as to imply that these operations are necessarily order dependent. Alternative ordering will be appreciated based on this description. Further, not all operations may necessarily be present in each aspect provided herein.

As used in this application, “or” is intended to mean an inclusive “or” rather than an exclusive “or”. Further, an inclusive “or” may include any combination thereof (e.g., A, B, or any combination thereof). In addition, “a” and “an” as used in this application are generally construed to mean “one or more” unless specified otherwise or clear from context to be directed to a singular form. Additionally, at least one of A and B and/or the like generally means A or B or both A and B. Further, to the extent that “includes”, “having”, “has”, “with”, or variants thereof are used in either the detailed description or the claims, such terms are intended to be inclusive in a manner similar to the term “comprising”.

Further, unless specified otherwise, “first”, “second”, or the like are not intended to imply a temporal aspect, a spatial aspect, an ordering, etc. Rather, such terms are merely used as identifiers, names, etc. for features, elements, items, etc. For example, a first channel and a second channel generally correspond to channel A and channel B or two different or two identical channels or the same channel. Additionally, “comprising”, “comprises”, “including”, “includes”, or the like generally means comprising or including, but not limited to.

It will be appreciated that various of the above-disclosed and other features and functions, or alternatives or varieties thereof, may be desirably combined into many other different systems or applications.

1. A computer-implemented method for inferring human mental states through a multimodal theory-of-mind (ToM) framework, the method comprising:
receiving a video dataset and a textual dataset associated with an environmental scene;
converting at least a portion of the video dataset and the textual dataset into symbolic representations of states, actions, and hypotheses related to an agent's goals and beliefs;
generating, based on the symbolic representations, action-likelihood distributions via a language model based policy, wherein the language model based policy is obtained by combining a large pre-trained language model with a smaller post-trained language model through a weak-to-strong control mechanism;
applying a Bayesian inverse-planning procedure that generates posterior probabilities over multiple goal-belief hypotheses based on the action-likelihood distributions and the symbolic representations; and
outputting at least one inferred mental state of the agent by selecting from among the multiple goal-belief hypotheses.

2. The computer-implemented method of claim 1, wherein video frames of the video dataset and textual descriptions of the textual dataset are parsed and aligned in converting the portion of the video dataset and the textual dataset into the symbolic representations including at least one of object relationships, agent actions, and timestamps.

3. The computer-implemented method of claim 2, wherein applying the Bayesian inverse-planning procedure includes updating a belief distribution based on new observations from each video frame and each textual description, such that the belief distribution is a probability over possible states of the environmental scene of the agent.

4. The computer-implemented method of claim 1, wherein generating the action-likelihood distributions via the language model based policy includes:
utilizing a policy distribution from the large pre-trained language model across a plurality of inference steps;
utilizing a post-trained small language model distribution from the smaller post-trained language model calibrated to domain specific data; and
reweighting the policy distribution from the large pre-trained language model based on a ratio of the post-trained small language model distribution over a naive small language model distribution.

5. The computer-implemented method of claim 4, wherein the ratio at each of the plurality of inference steps is normalized.

6. The computer-implemented method of claim 1, wherein the smaller post-trained language model is post-trained on a specialized dataset of at least one of human actions, states, and corresponding beliefs or goals.

7. The computer-implemented method of claim 1, wherein applying the Bayesian inverse-planning procedure includes computing a product of the action-likelihood distributions and belief updating factors across a plurality of time steps.

8. The computer-implemented method of claim 1, further including comparing two or more candidate hypotheses from among the multiple goal-belief hypotheses and determining which of the candidate hypothesis has a higher cumulative log-likelihood given an observed sequence of states and actions.

9. The computer-implemented method of claim 1, wherein the method is performed in real time during an interactive scenario, such that the at least one inferred mental state is utilized to autonomously control an autonomous system.

10. A system for inferring human mental states through a multimodal theory-of-mind (ToM) framework comprising:
a processor; and
a memory storing instructions when executed by the processor cause the processor to:
receive a video dataset and a textual dataset associated with an environmental scene;
convert at least a portion of the video dataset and the textual dataset into symbolic representations of states, actions, and hypotheses related to an agent's goals and beliefs;
generate, based on the symbolic representations, action-likelihood distributions via a language model based policy, wherein the language model based policy is obtained by combining a large pre-trained language model with a smaller post-trained language model through a weak-to-strong control mechanism;
apply a Bayesian inverse-planning procedure that generates posterior probabilities over multiple goal-belief hypotheses based on the action-likelihood distributions and the symbolic representations; and
output at least one inferred mental state of the agent by selecting from among the multiple goal-belief hypotheses.

11. The system of claim 10, wherein video frames of the video dataset and textual descriptions of the textual dataset are parsed and aligned in converting the portion of the video dataset and the textual dataset into the symbolic representations including at least one of object relationships, agent actions, and timestamps.

12. The system of claim 11, wherein applying the Bayesian inverse-planning procedure includes updating a belief distribution based on new observations from each video frame and each textual description, such that the belief distribution is a probability over possible states of the environmental scene of the agent.

13. The system of claim 10, wherein generating the action-likelihood distributions via the language model based policy includes:
utilizing a policy distribution from the large pre-trained language model across a plurality of inference steps;
utilizing a post-trained small language model distribution from the smaller post-trained language model calibrated to domain specific data; and
reweighting the policy distribution from the large pre-trained language model based on a ratio of the post-trained small language model distribution over a naive small language model distribution.

14. The system of claim 13, wherein the ratio at each of the plurality of inference steps is normalized.

15. The system of claim 10, wherein the smaller post-trained language model is post-trained on a specialized dataset of at least one of human actions, states, and corresponding beliefs or goals.

16. The system of claim 10, wherein applying the Bayesian inverse-planning procedure includes computing a product of the action-likelihood distributions and belief updating factors across a plurality of time steps.

17. The system of claim 10, wherein the processor is further configured to compare two or more candidate hypotheses from among the multiple goal-belief hypotheses and determine which of the candidate hypothesis has a higher cumulative log-likelihood given an observed sequence of states and actions.

18. The system of claim 10, wherein the system is configured to function in real time during an interactive scenario, such that the system utilizes the at least one inferred mental state to autonomously control an operatively connected autonomous system.

19. A non-transitory computer readable storage medium storing instructions that when executed by a computer, which includes a processor performs a method, the method for inferring human mental states through a multimodal theory-of-mind (ToM) framework comprising:
receiving a video dataset and a textual dataset associated with an environmental scene;
converting at least a portion of the video dataset and the textual dataset into symbolic representations of states, actions, and hypotheses related to an agent's goals and beliefs;
generating, based on the symbolic representations, action-likelihood distributions via a language model based policy, wherein the language model based policy is obtained by combining a large pre-trained language model with a smaller post-trained language model through a weak-to-strong control mechanism;
applying a Bayesian inverse-planning procedure that generates posterior probabilities over multiple goal-belief hypotheses based on the action-likelihood distributions and the symbolic representations; and
outputting at least one inferred mental state of the agent by selecting from among the multiple goal-belief hypotheses.

20. The non-transitory computer readable storage medium of claim 19, wherein generating the action-likelihood distributions via the language model based policy includes:
utilizing a policy distribution from the large pre-trained language model across a plurality of inference steps;
utilizing a post-trained small language model distribution from the smaller post-trained language model calibrated to domain specific data; and
reweighting the policy distribution from the large pre-trained language model based on a ratio of the post-trained small language model distribution over a naive small language model distribution.
